(19) United States
(12) Patent Application Publication
(10) Pub. No.: US 2017/0283548 A1
(43) Pub. Date: Oct. 5, 2017

(54) POLY (E-CAPROLACTONE)-ETHOXYLATED FATTY ALCOHOL COPOLYMERS

(71) Applicant: KING SAUD UNIVERSITY, RIYADH (SA)

(72) Inventors: ZIYAD BINKHATHLAN, RIYADH (SA); ABDULLAH H. ALOMRANI, RIYADH (SA); AWS ALSHAMSAN, RIYADH (SA); IBRAHIM I. ALJUFFALI, RIYADH (SA); RAISUDDIN ALI, RIYADH (SA)

(21) Appl. No.: 15/084,441

(22) Filed: Mar. 29, 2016

(51) Int. Cl. C08G 63/82; A61K 47/34; C08G 63/08; A61K 9/107

(52) U.S. Cl. CPC C08G 63/823; A61K 9/1075; A61K 47/34; C08G 63/08

(57) ABSTRACT

The poly(ε-caprolactone)-ethoxylated fatty acid copolymers are block copolymers including ε-caprolactone units and ethoxylated fatty alcohol units, the block copolymer having the structural formula:

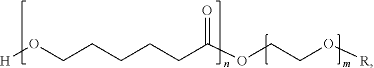

where n and m are integers greater than 0 and R is an alkyl group. The block copolymer is prepared by polymerizing ε-caprolactone and an ethoxylated fatty alcohol in the presence of a catalyst, such as stannous octoate. The block copolymers have potential as delivery systems for various payloads, such as, but not limited to, lipid soluble drugs and diagnostic agents.

### BACKGROUND OF THE INVENTION

### 1. Field of the Invention

The present invention relates to polymers, and particularly to nanocarrier-forming poly(ε-caprolactone)-ethoxylated fatty alcohol copolymers.

### 2. Description of the Related Art

Ethoxylated fatty alcohols that are sold under the unregistered trademark Brij™ by the Croda group of companies are non-ionic surfactants widely used with various drug delivery systems. The presence of PEG (polyethylene glycol) in the molecule extends the circulation time of the drug in plasma, while fatty acid enhances the solubility of the fat-soluble drug. Several derivatives of these ethoxylated fatty alcohols with different PEG chain lengths and fatty alcohols have been studied. Depending on the length of the fatty alcohol and the length of PEG used, the ethoxylated fatty alcohols products have hydrophilic-lipophilic balance (HLB) values in the range of 4-18 and critical micelle concentrations (CMC) in the micro-molar range.

In recent years, the ethoxylated fatty acids have been extensively used in the pharmaceutical industry. Several of these ethoxylated fatty alcohols products have been used as absorption enhancers, emulsifiers, solubilizers, permeation enhancers and stabilizers. Some derivatives of these ethoxylated fatty alcohols have also been used as inhibitors of P-gp (p-glycoprotein) to increase the oral bioavailability of P-gp substrates.

Poly(ε-Caprolactone) (PCL) is a biodegradable and biocompatible polyester that has been extensively studied for controlled drug delivery and tissue engineering applications. It is compatible with a wide range of drugs, which allows homogenous drug distribution in the polymer matrix. Moreover, PCL exhibits a long degradation time, leading to sustained drug release that could last for months. PCL has flexible mechanical properties that are suitable for medical applications, including drug delivery. Compared to other core-forming blocks in the poly(ester) category, such as PLGA (poly(lactic-co-glycolic acid)) and PLA (poly D-lactic acid), PCL is more hydrophobic, which makes it more compatible with hydrophobic drugs. The hydrophobicity of PCL has pushed the CMC of PEG-b-PCL to extremely low concentration in 100 nM range. PCL-based polymeric micelles have been successfully used to deliver a variety of lipophilic drugs, including P-gp inhibitors, such as Cyclosporine A and valspodar, and some of the chemotherapeutic agents, such as doxorubicin and paclitaxel. Thus, PCL is an important synthetic biomedical material with controlled biodegradability, which has already been approved by the USFDA and the European Medicines Agency (EMA) for clinical use as implants/drug delivery system.

Thus, poly(ε-caprolactone)-ethoxylated fatty alcohol copolymers are desired.

### SUMMARY OF THE INVENTION

The poly(ε-caprolactone)-ethoxylated fatty acid copolymers are block copolymers including ε-caprolactone units and ethoxylated fatty alcohol units. The copolymers include compounds of Formula I, as shown below:

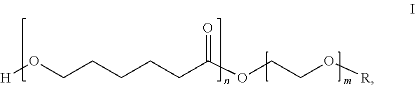

where n and m are integers greater than 0 and R is an alkyl group.

A method of preparing self-assembled nanocarriers comprises dissolving a block copolymer of Formula I in an organic solvent to form a solution; adding the solution drop-wise into distilled water; and evaporating the organic solvent to form self-assembled nanocarriers.

These and other features of the present invention will become readily apparent upon further review of the following specification and drawings.

### BRIEF DESCRIPTION OF THE DRAWINGS

FIG. 1 is a reaction formula for synthesis of an exemplary poly(ε-caprolactone)-ethoxylated fatty alcohol copolymer according to the present invention.

FIG. 2A is a representative ¹H-NMR spectra of Brij™ S100 (polyoxyethylene (100) stearyl ether).

FIG. 2B is representative ¹H-NMR spectra of PCL₈₈-b-Brij™-S100 block copolymer.

FIG. 3A is an overlay of Gel Permeation Chromatography (GPC) chromatograms of Brij™ L23, PCL₁₈-Brij™ L23, and PCL₃₅-Brij™ L23 block copolymers.

FIG. 3B is an overlay of Gel Permeation Chromatography (GPC) chromatograms of Brij™ S100, PCL₄₄-Brij™ S100, and PCL₈₈-Brij™ S100 block copolymers.

FIG. 4 shows representative X-ray diffraction (XRD) spectra of Brij™ 35 (Brij™ L23), Brij™ S100, and exemplary PCL-b-Brij™ block copolymers.

FIG. 5 is an overlay of Differential Scanning calorimetry (DSC) thermograms of Brij™ 35 (Brij L23), Brij™ S100, and PCL-b-Brij™ block copolymers.

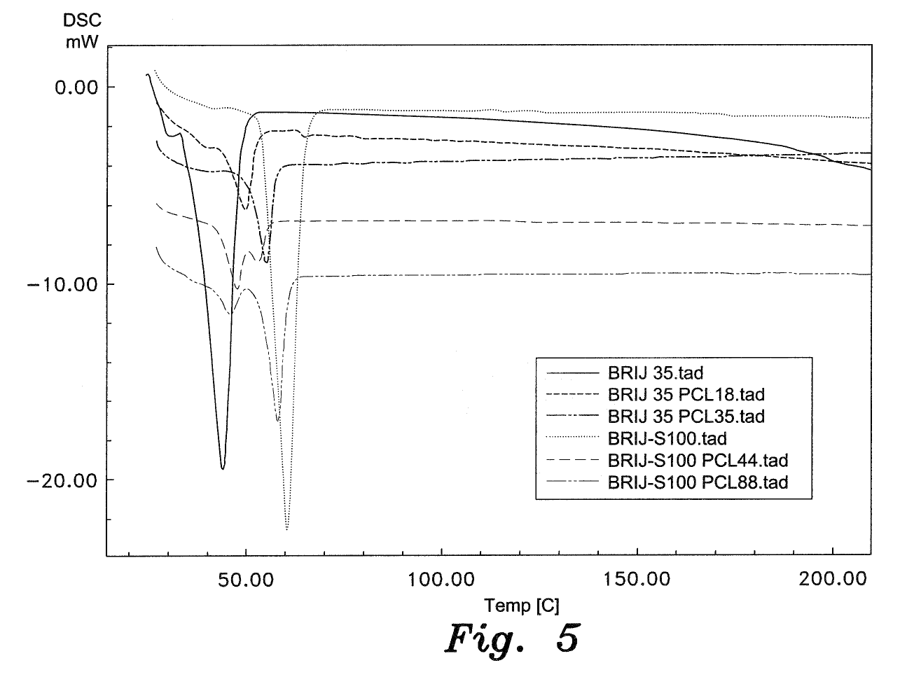

FIG. 6 are FTIR spectra of exemplary Brij™ fatty alcohols and PCL-b-Brij™ block copolymers.

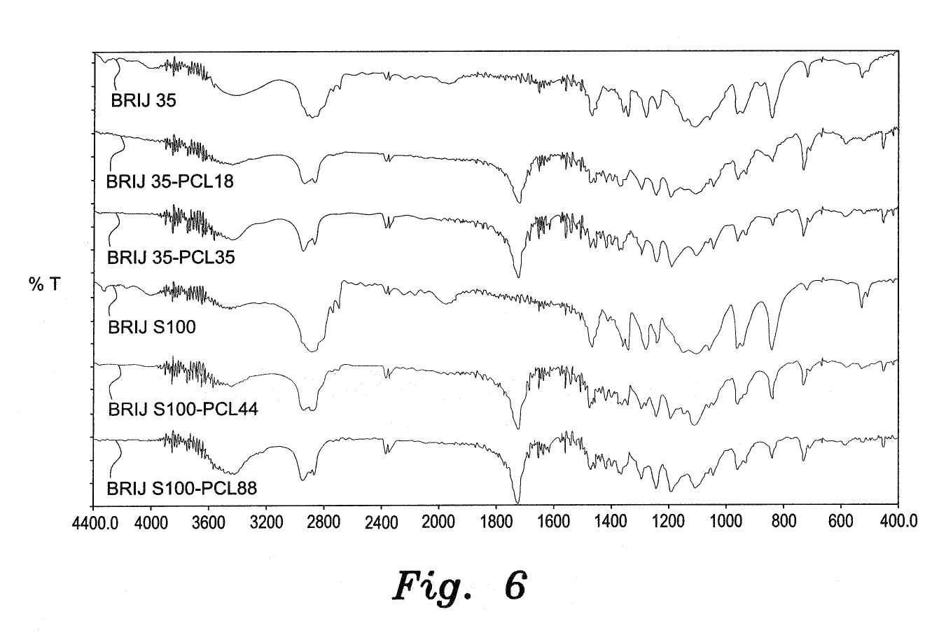

Similar reference characters denote corresponding features consistently throughout the attached drawings.

### DETAILED DESCRIPTION OF THE PREFERRED EMBODIMENTS

The poly(ε-caprolactone)-ethoxylated fatty alcohol copolymers are block copolymers including: ε-caprolactone units and ethoxylated fatty alcohol units. For example, the copolymers can be block copolymers including ε-caprolactone units and polyoxyethylene stearyl ether units. The copolymers include compounds of Formula I, as shown below:

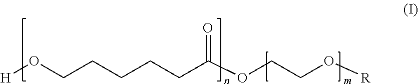

wherein n and m are integers greater than 0 and R is an alkyl group.

In the above formula, n can be from 1 to 100, m can be from 10 to 100, and R can be an alkyl group having 12-20 carbon atoms. For example, R can be an alkyl group having 18 carbon atoms. The block copolymer can possess an average molecular weight of at least 1500 Daltons, preferably 1500 to 100,000 Daltons.

The block copolymer may be prepared by polymerizing (i) ε-caprolactone and (ii) ethoxylated fatty alcohol in the presence of a catalyst, where the polymerization reaction occurs at a temperature of about 140-150° C. for about 4 hours, and where the catalyst is stannous octoate.

For example, ethoxylated fatty alcohols sold under the trade name of Brij™, ε-caprolactone and stannous octoate can be added to a previously flamed ampoule, purged with nitrogen, and sealed under vacuum. The polymerization reaction can be conducted at a temperature of about 140-150° C. for about 4 hours to about 5 hours. The reaction can be terminated by cooling the product to room temperature. The produced block copolymer of Formula I typically has a molecular weight in the range of about 1500 Daltons to about 100,000 Daltons, or even higher.

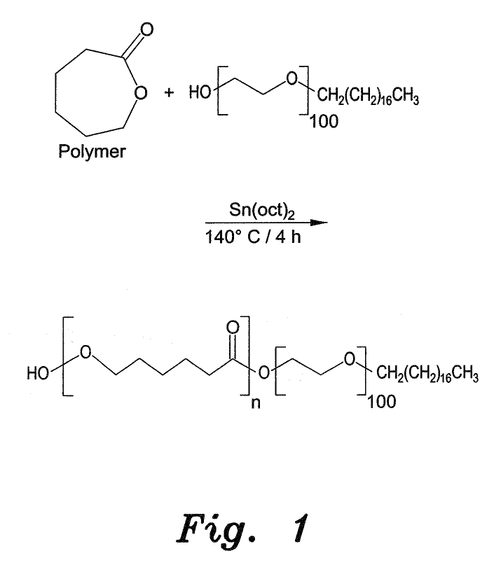

The PCL-Brij™ block copolymer with various PCL/Brij™ ratios were synthesized by ring-opening bulk polymerization of ε-caprolactone using Brij™ fatty alcohols as an initiator and stannous octoate as a catalyst, as depicted schematically in FIG. 1.

The copolymers of Formula I are biodegradable and can be used for drug delivery. For example, self-assembled nanocarriers including the copolymers of Formula I can be prepared by dissolving a block copolymer of Formula I in an organic solvent to form a solution; adding the solution drop-wise into distilled water; and evaporating the organic solvent to form self-assembled nanocarriers. The nanocarriers have a mean diameter of about 50 nm to about 300 nm.

The PCL-Brij™ copolymers improve the kinetic and thermodynamic stability of the micelles formed by reducing the CMC, and therefore the micelles become more resistant to dilution compared to the unmodified Brij™. Additionally, the PCL-Brij™ copolymers enhance the hydrophobic drug loading capacity inside the core of the micelles and further enhance the solubility and permeability of hydrophobic drugs across cellular membranes, thereby controlling the rate of drug release from the micelles/nanocarriers. Moreover, the PCL-Brij™ copolymers have potential use as a targeted delivery system for drugs and diagnostic agents, as well as potential use as a delivery system for treatment of multi-drug resistant tumors.

The following examples will further illustrate the synthetic processes of making the poly(ε-caprolactone)-ethoxylated fatty alcohol copolymers and the nanocarriers.

### Example 1

### Synthesis of PCL-Brij™ Copolymers

PCL-Brij™ copolymer with various PCL/Brij™ ratios were synthesized by ring opening bulk polymerization of ε-caprolactone using a Brij™ fatty alcohol as an initiator and stannous octoate as a catalyst, as illustrated in FIG. 1. Other catalysts, such as bidentate sulfonamide zinc complex, can also be used for this coupling reaction. The Brij™ fatty alcohol, ε-caprolactone, and stannous octoate were added to a previously flamed 10 mL ampoule, nitrogen purged, then sealed under vacuum. The polymerization reactions were allowed to proceed for 4-5 h at 140° C. in an oven. The reaction was terminated by cooling the product to room temperature. Table 1 shows the representative list of synthesized PCL-Brij™ copolymers.

TABLE 1
Synthesized Copolymers

| | Theor. | | | |
|---|---|---|---|---|
| | MW | | | |
| Block copolymer ^{a} | (g/mol) | Mₙ (g/mol)^{b} | Mₙ (g/mol)^{c} | PDI^{d} |
| PCL₁₈-b-Brij ™ L23 | 3,250 | 3,200 | 4,800 | 1.05 |
| PCL₃₅-b-Brij ™ L23 | 5,200 | 5,350 | 7,300 | 1.41 |
| PCL₄₄-b-Brij ™ S100 | 9,700 | 9,500 | 9,000 | — |
| PCL₈₈-b-Brij ™ S100 | 14,650 | 14,650 | 11,100 | 1.21 |

In Table 1, the superscript “a” represents the polymerization degree of each block determined by ¹H NMR; the superscript “b” represents the number-average molecular weight measured by ¹H NMR; the superscript “c” represents the number-average molecular weight measured by GPC; and the superscript “d” represents the polydispersity index (M_{w}/Mₙ) determined by GPC. The ethoxylated fatty alcohol sold under the trademark Brij™ L23, and referenced above in Table 1, refers to polyoxyethylene (23) lauryl ether. The ethoxylated fatty alcohol sold under the trademark Brij™ S100, and referenced above in Table 1, refers to polyoxyethylene (100) stearyl ether.

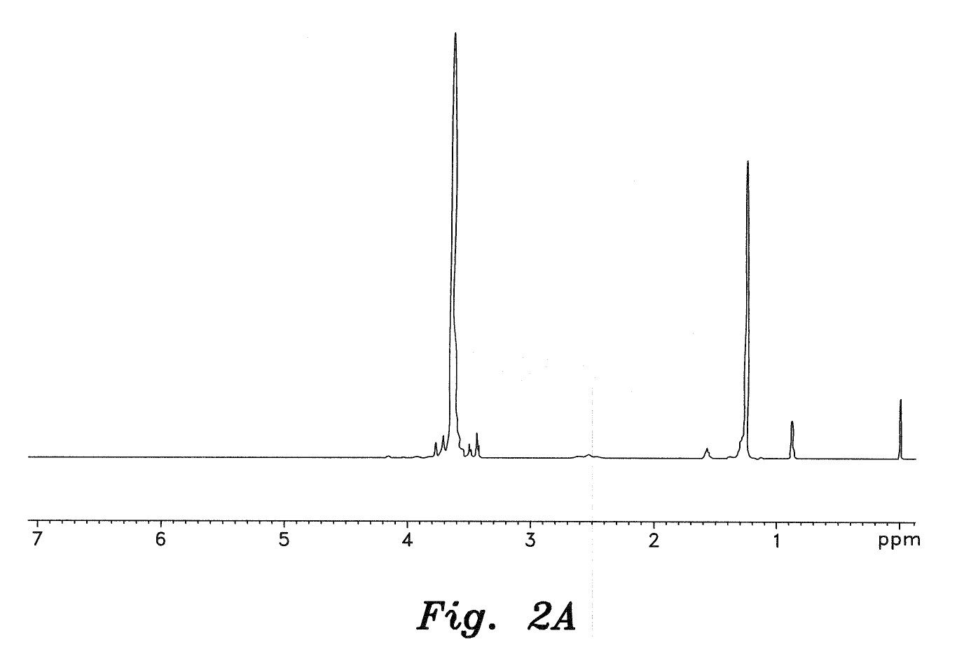

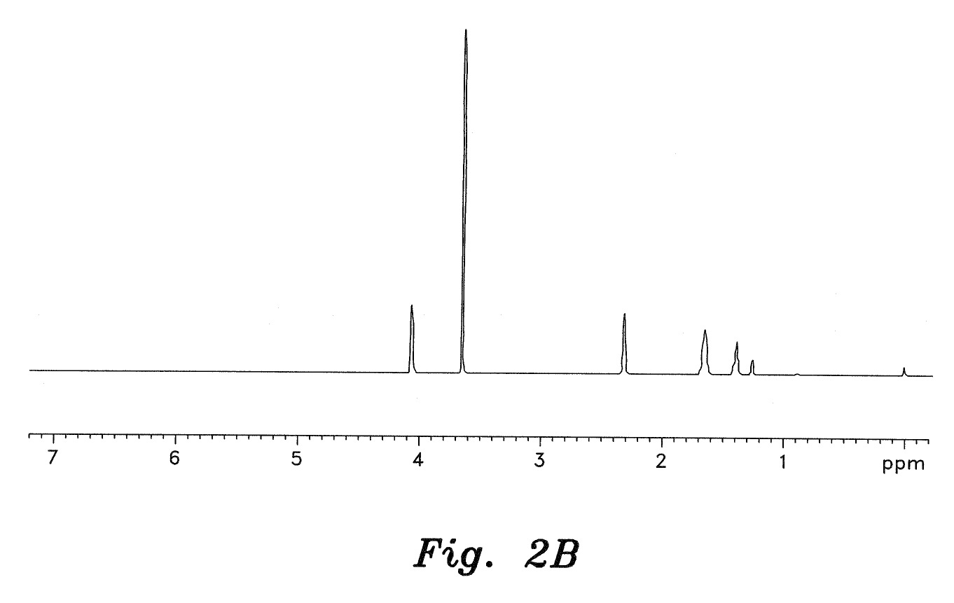

Table 1 displays the calculated Mₙ values of the synthesized PCL-Brij™ copolymers. The number average molecular weight of PCL-Brij™ copolymers formed was determined from ¹H NMR spectra by comparing the peak intensity of PEG (—CH₂CH₂O—, δ=3.65 ppm) to that of PCL (—OCH₂—, δ=4.07 ppm). The calculation used the integration area of the peaks of methylene protons of PCL at 4.07 ppm and of PEG at 3.65 ppm, respectively. The calculated Mₙ values obtained from GPC confirmed the NMR data. The polymerization reaction yielded PCL-b-Brij™ copolymers with a unimodal distribution, as confirmed by GPC. The representative ¹H NMR spectra of Brij™ S100 and PCL₈₈-b-Brij™ S100 copolymer are shown in FIGS. 2A and 2B, respectively.

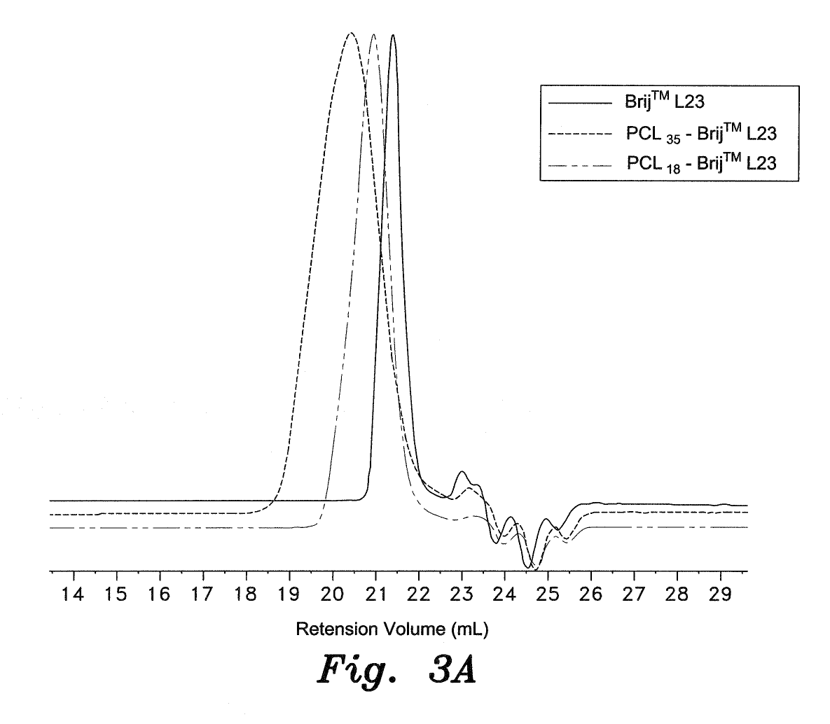

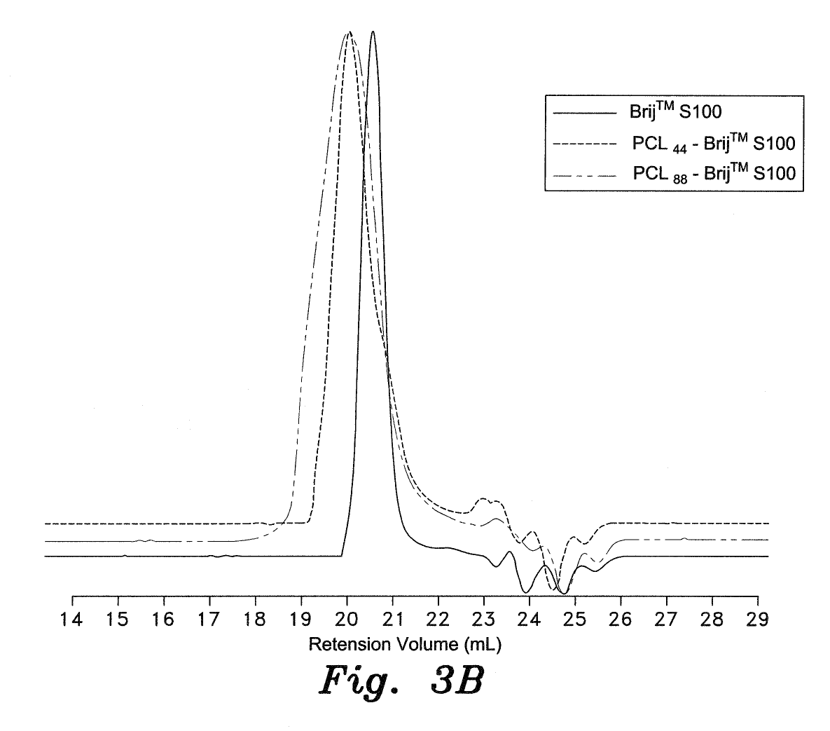

The weight and number average molecular weight, as well as the polydispersity of the prepared polymers, were assessed by gel permeation chromatography (GPC) (Viscotek TDA 305-040 Triple Detector Array, Viscotek Corp., Houston, Tex., USA). Samples (100 μL from 15 mg/mL polymer stock solutions in THF) were injected into an 8.0×300 mm Viscotek T6000M column (Viscotek Corp., Houston, Tex., USA) with guard column. The mobile phase was THF delivered at a flow rate of 1 ml/min. The calibration curve was established by using six polystyrene standards. GPC chromatograms of Brij™ and PCL-b-Brij™ copolymers are provided in FIGS. 3A and 3B.

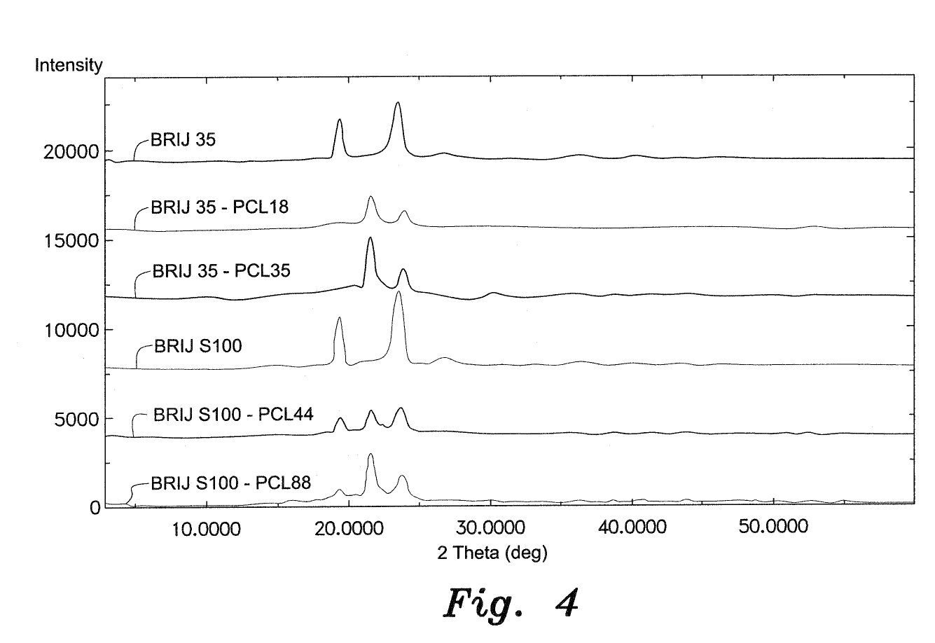

An X-ray Diffractometer was used to study the crystallinity state of the synthesized copolymers. Samples of the copolymers and the Brij™ fatty alcohol were loaded in the XRD instrument (automated Rigaku Ultima IV). The X-ray diffractogram of the investigated sample was collected using 2theta (2θ) scan axis mode, scan speed set at 0.5°/min, and covering scan range of 3.0-50.0 deg. The scanning process was performed at room temperature. FIG. 4 shows representative X-ray diffraction (XRD) spectra of representative Brij™ fatty alcohols35 (Brij™ L23), Brij™ S100, and exemplary PCL-b-Brij™ block copolymers synthesized according to the method herein described.

The thermograms of Brij™ and PCL-b-Brij™ were obtained using differential scanning calorimetry (DSC-60, Shimadzu, Japan). Samples (3-5 mg) were loaded in an aluminum pan and sealed with aluminum lids by a crimper. The sample was then thermally scanned against an empty aluminum pan with lid at a heating rate of 10° C./min and covering temperatures ranging from 25-200° C. Nitrogen purging at 40 ml/min was used during scanning. The TA-60WS thermal analysis software was used to calculate the thermal parameters of the scanned sample.

The FTIR spectra of the synthesized copolymers were obtained using an FTIR spectrophotometer (PerkinElmer, USA). A copolymer sample was ground with potassium bromide (spectroscopic grade) and compressed into a thin disk using hydraulic press before scanning from 4400 to 400 cm⁻¹.

### Example 2

### Assembly of PCL-Brij™ Block-Copolymers

Assembly of self-assembled nanocarriers was achieved by co-solvent evaporation, where PCL-Brij™ (30 mg) dissolved in acetone (0.5 mL) was added in a drop-wise manner (1 drop/15 s) to distilled water (3 mL) while stirring. The remaining acetone was removed by evaporation at room temperature under vacuum. Other organic solvents, such as tetrahydrofuran, acetonitrile and dimethyl oxide (DMSO), can also be used. Mean diameter and polydispersity of the self-assembled structures in aqueous media were defined by light scattering (Zetasizer™ Nano ZS, Malvern Instrument Ltd., UK). Typically, the average sizes of the nanocarrier is in the range of 50-300 nm, as shown in Table 2, but it could also be outside these ranges.

TABLE 2
Properties of self-assembled nanocarriers

| | | Poly dispersity |
|---|---|---|
| Block Copolymer^{a} | Size (nm)^{a} | (PD^{b}) |
| PCL₁₈-Brij ™ L23 | — | — |
| PCL₃₅-Brij ™ L23 | — | — |
| PCL₄₄-Brij ™ S100 | 197.4 | 0.117 |
| PCL₈₈-Brij ™ S100 | 163.5 | 0.257 |

In Table 2, the subscripts “a” and “b” represent mean diameter (Zₐᵥₑ) and polydispersity of unloaded nanocarriers estimated by the dynamic light scattering (DLS) technique, respectively. The conjugation of PCL with Brij™ is believed to increase micelle stability and drug solubilization. The biodegradability of these copolymers and their biocompatibilities with a large number of hydrophobic drugs make them suitable as carriers for various lipid-soluble drugs in drug delivery. The built-in P-glycoprotein (P-gp) inhibiting activity in some of the Brij™ molecules is believed to enhance the intestinal permeability of P-gp substrates and overcoming multi-drug resistance in cancer.

It is to be understood that the present invention is not limited to the embodiments described above, but encompasses any and all embodiments within the scope of the following claims.

1. A poly(ε-caprolactone)-ethoxylated fatty alcohol copolymer, comprising a block copolymer having at least one ε-caprolactone unit and ethoxylated fatty alcohol units, the block copolymer having the structural formula: 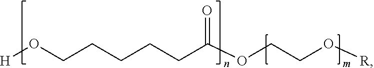
where n and m are integers greater than 0 and R is an alkyl group, and
wherein the poly(ε-caprolactone)-ethoxylated fatty alcohol copolymer of claim 1, wherein n is 1 to 100, m is 10 to 100, and R is an alkyl group having 12-18 carbon atoms.

2-3. (canceled)

4. The poly(ε-caprolactone)-ethoxylated fatty alcohol copolymer of claim 1, wherein the block copolymer has an average molecular weight of at least 3000 Daltons.

5. A method of making a poly(s-caprolactone)-ethoxylated fatty alcohol copolymer, comprising the steps of:
mixing ε-caprolactone and an ethoxylated fatty alcohol in a reaction vessel to form a reaction mixture;
adding a catalyst to the reaction mixture to catalyze polymerization; and
heating the reaction vessel to 140° C.

6. The method of making a poly(ε-caprolactone)-ethoxylated fatty alcohol copolymer according to claim 5, wherein said step of heating the reaction vessel to 140° C. comprises polymerizing the reaction mixture at a temperature of about 140° C. for between 4-5 hours.

7. The method of making a poly(ε-caprolactone)-ethoxylated fatty alcohol copolymer according to claim 5, further comprising the steps of:
purging the reaction vessel of nitrogen;
sealing the reaction vessel; and
polymerizing the reaction mixture under vacuum.

8. The method of making a poly(ε-caprolactone)-ethoxylated fatty alcohol copolymer according to claim 5, wherein said catalyst comprises stannous octoate.

9. The method of making a poly(ε-caprolactone)-ethoxylated fatty alcohol copolymer according to claim 5, wherein said catalyst comprises bidentate sulfonamide zinc complex.

10. The method of making a poly(ε-caprolactone)-ethoxylated fatty alcohol copolymer according to claim 5, wherein the ethoxylated fatty alcohol has a molecular weight between 1000 and 50,000 Daltons.

11. The method of making a poly(ε-caprolactone)-ethoxylated fatty alcohol copolymer according to claim 5, further comprising the step of cooling the reaction vessel to room temperature to terminate polymerization.

12. The method of making a poly(ε-caprolactone)-ethoxylated fatty alcohol copolymer according to claim 5, wherein the ethoxylated fatty alcohol comprises polyoxyethylene stearyl ether.

13. (canceled)

14. A method of preparing self-assembled nanocarriers, comprising the steps of:
dissolving a poly(ε-caprolactone)-ethoxylated fatty alcohol block copolymer in an organic solvent to form a solution;
adding the solution drop-wise into distilled water; and
evaporating the organic solvent to form self-assembled nanocarriers.

15. The method of preparing self-assembled nanocarriers according to claim 14, wherein the self-assembled nanocarriers have a mean diameter between 50 nm and 300 nm.

16. The method of preparing self-assembled nanocarriers according to claim 14, wherein the organic solvent comprises at least one solvent selected from the group consisting of acetone, tetrahydrofuran, acetonitrile, and dimethyl oxide.
